(19) United States
(12) Patent Application Publication
(10) Pub. No.: US 2009/0252816 A1
(43) Pub. Date: Oct. 8, 2009

(54) Inhalable Gaseous Medicament Based On Xenon And Nitrous Oxide

(75) Inventors: Jacques Abraini, Caen (FR); Marc Lemaire, Paris (FR)

Correspondence Address: AIR LIQUIDE;Intellectual Property, 2700 POST OAK BOULEVARD, SUITE 1800, HOUSTON, TX, 77056, US

(73) Assignee: AIR LIQUIDE SANTE (INRTERNATIONAL), Paris (FR)

(21) Appl. No.: 12/417,087

(22) Filed: Apr. 2, 2009

Related U.S. Application Data
(62) Division of application No. 10/563,278, filed on Oct. 6, 2006.

(30) Foreign Application Priority Data
Jul. 30, 2003 (FR) 0350383

(51) Int. Cl. A61K 33/00

(52) U.S. Cl. 424/718

(57) ABSTRACT

The invention concerns the use of a gaseous mixture containing gas xenon and gas nitrous oxide, and advantageously oxygen, for making all or part of an inhalable medicine for preventing or treating neurointoxication in a human. The xenon/nitrous oxide mixture of the invention acts on one or more cerebral receptors to reduce the release and/or the effects of dopamine, glutamate, serotonin, taurin, GABA, noradrenaline and/or any other neurotransmitter. The proportion by volume of xenon of the mixture ranges between 5 and 45%, and the proportion by volume of nitrous oxide ranges between 10 and 50%, the balance is preferably oxygen.

### CROSS REFERENCE TO RELATED APPLICATION

This present application is a divisional of U.S. patent application Ser. No. 10/563,278 which has a U.S. filing date of Oct. 6, 2006 and which is a §371 of International PCT Application PCT/FR2004/050352, filed Jul. 23, 2004.

### FIELD OF INVENTION

The invention relates to the use of a gaseous mixture containing xenon and nitrous oxide (N₂O) to manufacture all or part of an inhalable medicament for treating or preventing a pathology with a neurotoxic effect, i.e. neurointoxication, especially the neurotoxic effects of drugs or other addictive substances.

### BACKGROUND

In pathologies associated with the neurotoxic effects of addictive drugs, such as amphetamines, it is accepted that dopaminergic neurotransmission of nigrostriatal and mesolimbic origin participates in the psychostimulant and neurotoxic effects of these drugs.

However, recent studies by Del Arco et al., Neuropharmacology, 38: 943, 1999, have shown that the facilitating effects of amphetamines are not limited to dopaminergic neurotransmission.

Thus, in the striatum-nucleus accumbens septi complex, amphetamines induce not only an increase in dopamine release but also an increase in serotonin, taurine, γ-aminobutyric acid (GABA) and glutamate release.

It has been shown, particularly advantageously, that the specific inhibition of glutamate transporters makes it possible to reduce both hyperactivity (David, Thévenoux and Abraini, Neuropharmacology, 2001) and the increase in glutamate, but not in dopamine (Del Arco et al., Neuropharmacology 38: 943, 1999), following injection of amphetamines, thus suggesting a decisive role of glutamate in the psychostimulant effects of amphetamines.

Moreover, recent studies, performed in vitro, have shown that xenon and nitrous oxide (N₂O) can behave like antagonists with low affinity for the N-methyl-D-aspartate glutamatergic receptors (NMDA: Franks et al., Nature 396: 324, 1998; Jevtovic-Todorovic et al; Nature Med. 4: 460, 1998).

In addition, in the context of the study of the endogenous hyperalgesic opioid system in the negative placebo response, F. J. Lichtigfeld and M. A. Gillman, Intern. J. Neuroscience, 1989, vol. 49, pp. 71-74 conclude that the effect of nitrous oxide on weaning from alcohol is slightly better than the placebo effect, although, for more than 50% of the individuals, an identical positive effect was also found with the placebo.

However, the same authors add, in Nitrous Oxide and the Aws, p. 785, that the beneficial effect of nitrous oxide depends closely on its concentration, since anesthetic or preanesthetic concentrations are ineffective, or are even counterproductive in certain cases, an analgesic concentration being recommended.

Yet another publication from these authors, published in Intern. J. Neuroscience, 1994, Vol. 76, pp. 17-33, underlines the rapid and long-lasting psychotropic analgesic effects of nitrous oxide in the mechanism of weaning from alcohol.

In Postgrad. Med. J, Clinical Toxicology, 1990, 66, pp. 543-546, the same authors explain that the nitrous oxide concentrations may vary from less than 15% to more than 70% depending on the individual, as a function of his or her degree of alcohol dependency.

Moreover, document EP-A-1 158 992 teaches the use of xenon or of a mixture of xenon with oxygen, nitrogen or air to treat neurointoxications. However, the use of xenon or of the mixtures described by said document is not entirely satisfactory in practice, especially due to the appearance of toxicity for certain xenon contents and given the high cost of this compound.

Moreover, U.S. Pat. No. 6,274,633 teaches the use of xenon as an NMDA receptor antagonist compound assumed to be involved in neurotoxicity and neuronal cell death caused by certain diseases or ischemic hypoxia or following a heart attack, in particular.

In addition, EP-A-861 672 proposes inhalable gaseous mixtures based on oxygen and on several possible gases, including xenon.

Finally, FR-A-2 596 989 proposes gaseous mixtures based on nitrous oxide and oxygen, which may possibly contain xenon or other gases, as radiosensitizing products, which may especially be used in cancer radiotherapy.

The present invention falls within this context and is directed toward improving the existing inhalable medicaments intended for effectively preventing or treating a state of addiction in humans, i.e. any condition, disorder or pathology associated with neurotoxic effects, in particular the neurotoxic effects of addictive drugs.

### SUMMARY OF THE INVENTION

The solution of the invention thus relates to the use of a gaseous mixture containing xenon (Xe) gas and nitrous oxide (N₂O) gas to manufacture all or part of an inhalable medicament for preventing or treating a neurointoxication in man, the volume proportion of xenon being between 5% and 45% and the volume proportion of nitrous oxide being between 10% and 50%.

### BRIEF DESCRIPTION OF THE FIGURES

FIG. 1 illustrates the effects of nitrous oxide at 50 vol % and 75 vol % (with the remainder of oxygen) on sensitization to d-amphetamine.

FIG. 2 illustrates the effects of xenon at 50% and 75% on sensitization to d-amphetamine.

FIG. 3 illustrates the effects of nitrous oxide on the increase in dopamine release in the nucleus accumbens septi induced with d-amphetamine.

### DETAILED DESCRIPTION OF THE INVENTION

The present invention relates to the use of a gaseous mixture containing xenon gas and nitrous oxide gas in order to manufacture all or part of an inhalable medicament for preventing or treating neurointoxication in man.

Depending on the case, the use of the invention may include one or more of the following technical characteristics:
the neurointoxication results from a cerebral excess of one or more neurotransmitters,
the mixture containing xenon and nitrous oxide acts on at least one cerebral receptor so as to reduce the effects and/or the release of dopamine, glutamate, serotonin, taurine, GABA, noradrenalin and/or any other neurotransmitter,
the volume proportion of xenon is between 20% and 40% and the volume proportion of nitrous oxide is between 10% and 40%,
the volume proportion of xenon is between 20% and 32% and the volume proportion of nitrous oxide is between 20% and 40%, and preferably the volume proportions of xenon and of nitrous oxide are each about 30%,
the volume proportion of xenon is between 10% and 20% and the volume proportion of nitrous oxide is between 40% and 50%, and preferably the volume proportion of xenon is about 16% and the volume proportion of nitrous oxide is about 50%,
the medicament also contains oxygen, an oxygen/nitrogen mixture or air, and the gaseous mixture preferably consists of xenon and nitrous oxide, the remainder being oxygen,
the neurointoxication is of the type giving rise to a state of addiction, i.e. a condition, disorder or pathology associated with the neurotoxic effects of a drug, molecule or substance generating an addiction, a dependency and/or a habit in man or animals. The addictive substance, drug or molecule is chosen from the group formed by amphetamines and derivatives thereof, cocaine, tobacco, alcohol and cannabis, or any other similar or analogous drug,
the inhalable medicament is packaged at a pressure of from 2 bar to 350 bar and preferably between 2 bar and 200 bar,
the medicament is ready-to-use, i.e. it may be administered to the patient directly without being prediluted.

The invention also relates to a gaseous inhalable medicament containing from 5% to 35% by volume of xenon and from 10% to 50% by volume of nitrous oxide, and possibly oxygen.

Depending on the case, the gaseous mixture of the invention may include one or more of the following technical characteristics:
it consists of from 5% to 32% by volume of xenon, from 10% to 50% of nitrous oxide, and the remainder is oxygen,
it consists of from 20% to 32% by volume of xenon and from 20% to 40% of nitrous oxide, the remainder being oxygen, and the volume proportions of xenon and of nitrous oxide are each preferably about 30%,
it consists of from 10% to 20% by volume of xenon and from 45% to 50% of nitrous oxide, the remainder being oxygen, and preferably the volume proportion of xenon is about 16% and the volume proportion of nitrous oxide is about 50%.

In other words, the idea forming the basis of the present invention is thus that the NMDA receptor antagonist properties of xenon and of nitrous oxide may be used, in a combined or synergistic manner, for their neuroprotective nature in the prevention and/or treatment of the conditions or disorders associated with neurotoxic effects, in particular the neurotoxic effects of addictive drugs, such as amphetamines and derivatives thereof, cocaine, tobacco, alcohol, cannabis or any other dependency-generating substance, especially all or part of an inhalable gaseous medicament.

In general, the medicament according to the invention may be administered to the patient via his upper respiratory pathways, i.e. by inhalation via the nose and/or the mouth, using a suitable administration device comprising a patient respiratory interface, such as a respiratory mask or tracheal probe, one or more feed pipes serving to convey the gaseous medicament from a source containing said medicament to the interface, and a medical ventilator serving to deliver and/or extract the patient's gas.

The invention also relates to a method for preventing or treating a neurointoxication in a human patient, in which a gaseous mixture containing xenon gas and nitrous oxide gas is administered by inhalation to said patient, the volume proportion of xenon in said gaseous mixture being between 5% and 45% and the volume proportion of nitrous oxide being between 10% and 50%.

### Examples

### Demonstration of the Neuroprotective Potential of Xenon and of Nitrous Oxide

In order to evaluate the neuroprotective potential of xenon and of nitrous oxide gas, the antagonist properties of which on the glutamatergic receptors of NMDA type have recently been demonstrated, on sensitization to amphetamines, behavioral, neurochemical and histological studies were performed as described below.

Male Sprague-Dawley rats weighing about 250 g were used in the experiments.

In the tests, the d-amphetamine sensitization protocol and the nitrous oxide and xenon treatment tests were as follows.

15 groups of animals (of 7 or 8 animals each) were used, including 10 groups during the actual sensitization studies and 5 other groups during the histological studies of the neurons of the posterior and retrosplenial cingulate cortices.

The animals were injected intraperitoneally (i.p.) for 3 consecutive days from D1 to D3 with d-amphetamine (Amph; 1 mg/ml/kg) or, depending on the case, with a saline solution (1 ml/kg) for the control animals.

After each injection, the rats were immediately placed for 3 hours in a closed chamber, 100 liters in volume, flushed in dynamic regime with a constant flow rate of 5 l.min⁻¹, either with air (group 1: saline; group 2: Amph), or with 50% by volume of nitrous oxide (group 3: saline; group 4: Amph) or 75% (group 5: saline; group 6: Amph), or with 50% by volume of xenon (group 7: saline; group 8: Amph) or 75% (group 9: saline, group 10: Amph); the rest of the mixtures (remainder to 100%) being oxygen.

In order to identify the possible neurotoxic potential of repeated exposure (3 hours per day for 3 hours) to nitrous oxide or to xenon on the posterior and retrosplenial cingulate cortices, 5 additional groups of animals were pretreated, according to a protocol identical to that defined above, by administration of a saline solution, and then exposed either to air (group 11) or to 50% or 75% nitrous oxide (groups 12 and 13) or to 50% or 75% xenon (groups 14 and 15).

The locomotor activity of the animals of groups 1 to 10 was evaluated on D6, after an i.p. injection of a saline solution (1 ml/kg), and on D7 after an i.p. injection of d-amphetamine (1 mg/ml/kg). The locomotor activity of the animals in response to these injections was recorded using actimetry cages with photoelectric cells (Imétronic, Pessac. France).

Moreover, neurochemical studies, in addition to the above histological and behavioral studies, were performed on slices of the brains of these rats in order to identify the mechanisms of the action of nitrous oxide and of xenon, and in order to evaluate the neurotoxic potential of nitrous oxide and xenon.

To do this, after treatment, the animals were sacrificed on D8 by decapitation under general anesthesia with halothane, and the cranium was then immediately placed in a paraformaldehyde solution for one week. The brain was removed, coated with paraffin and sectioned into frontal slices of 4 μm mounted on gelatinized slides and stained with a hemalun-eosin-saffron solution. The posterior and retrosplenial cingulate cortices were analyzed using an optical microscope (×400).

In addition, the preparation of the slices of nucleus accumbens septi was performed as follows. The animals were decapitated under mild anesthesia with halothane and the brain was then rapidly removed. Frontal sections of 300 μm, corresponding to an anteriority of +0.70/1.20 mm (according to Bregma, Paxinos and Watson, 1998) were taken using a chopper (Mickie Laboratory Engineering Company, Gomshall, Surrey, UK). The brain slices were placed for recovery in a buffered saline solution with a temperature of 3-4° C. for at least 1 hour before use for neurochemical study.

The measurement of the dopamine release was performed by the technique of normal differential pulse voltammetry using a single-fiber carbon electrode 10 μm in diameter and 250 μm long (CFN10-250; World Precision Instruments, Aston, Stevenage, Hertfordshire, UK). The electrochemical treatment enabling this type of electrode to be made sensitive to dopamine consisted in applying a continuous current of −1.5 V into a phosphate-buffered saline solution for 20 seconds, followed by a triangular current of +2.6 V also for 20 seconds on the working electrode (Brazell et al., 1987). Under these conditions, the dopaminergic signal appears at a potential of +100 mV.

The rat brain slices were then placed in an organ tank and infused with artificial cerebrospinal fluid having the composition: NaCl 118 mM, MgCl₂ 1.18 mM, KCl 4.9 mM, NaH₂PO₄ 1.25 mM, CaCl₂ 1.25 mM, NaHCO₃ 3.6 mM, d-glucose 10 mM, HEPES 30 mM, pH 7.4, the temperature of which was maintained at 34±1° C. using a temperature controller (Delta 4 Culture Dish Controller, Bioptechs, Butler, Pa., USA). The electrode was placed under microscopic control (microscope EFN 600, Nikon, Paris, France), 100 μm from the anterior commissure, using an optical micrometer incorporated into the microscope, and then fully descended into the nucleus accumbens septi, at an angle of 45°, and connected to the Biopulse polarograph set in normal differential pulse voltammetry mode with the following parameters: scanning potential −150+350 mV; scanning time 0.4 s, scanning amplitude 4 mV, for a scanning speed of 10 mV.s⁻¹, 40 ms measuring pulse; 70 ms measuring prepulse; 30 mV measuring amplitude.

The dopaminergic hyperstimulation was induced by adding d-amphetamine to the infusion liquid. Medical air, nitrous oxide or xenon was dissolved, to saturation, before use in the infusion liquid, the pH of which was readjusted to 7.4.

The d-amphetamine (d-amphetamine sulfate, ref. A5880) was acquired after authorization from the stupefacients and psychotropics unit of the Agence Française de Sécurité Sanitaire des Produits de Santé from Sigma-Aldrich (Illkirch, France).

The medical air, nitrous oxide and xenon were supplied by Air Liquide Santé International (Paris, France). The mixtures based on nitrous oxide, oxygen and/or xenon were prepared using calibrated flow meters also supplied by Air Liquide Santé International.

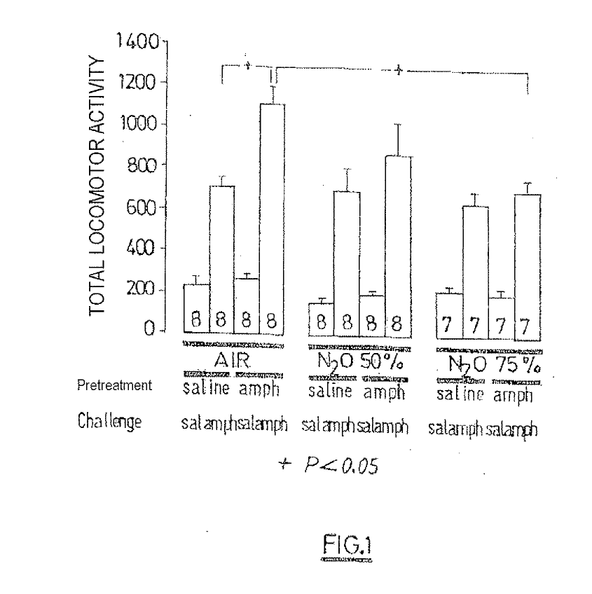

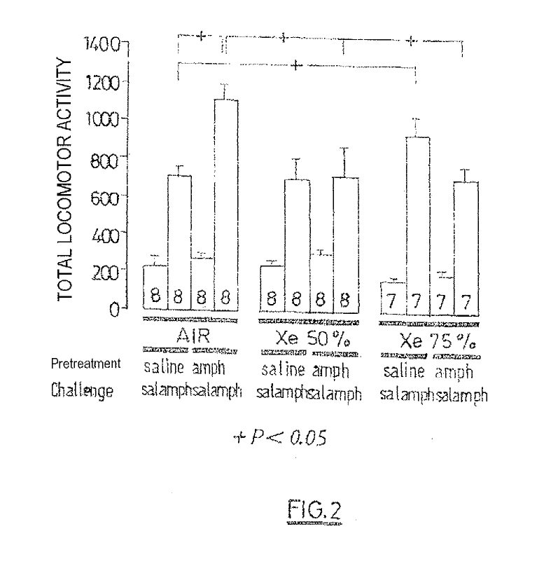

The results obtained, given in the attached FIGS. 1 and 2, are expressed as the mean±standard error. The comparison of the groups was performed by means of nonparametric tests: Kruskall-Wallis analysis of variance, followed in the event of a significant result by the Mann-Whitney U test.

More specifically, for the behavioral aspect, the left-hand histograms in FIGS. 1 and 2 illustrate the sensitization process induced by the repeated administration of d-amphetamine, since:
FIG. 1 represents the effects of nitrous oxide at 50 vol % and 75 vol % (remainder oxygen) on sensitization to d-amphetamine; and
FIG. 2 illustrates the effects of xenon at 50% and 75% on sensitization to d-amphetamine.

It may be seen in these figures that the repeated injection of d-amphetamine produces an increase in locomotor activity induced by the acute injection of d-amphetamine, such that the locomotor activity of the animals pretreated with d-amphetamine (amph in the figure) appears significantly higher than that of the control animals pretreated using a saline solution (saline in the figure), during the test with d-amphetamine performed on D7 (P<0.05).

On the other hand, a repeated injection from D1 to D3 of d-amphetamine produces no significant difference in locomotor activity between the animals pretreated with d-amphetamine and the control animals in response to the saline test performed on D6.

As regards FIG. 1, it is found that, under the above experimental conditions, exposure to nitrous oxide immediately after pretreatment with d-amphetamine induces dose-dependent blocking of the sensitization process.

Thus, the locomotor activity of the animals pretreated with d-amphetamine and with nitrous oxide at 50 vol % induced by the test with d-amphetamine performed on D7 does not appear significantly different from the motor activity of the rats pretreated with a saline solution and nitrous oxide at 50 vol %, or from that of the animals pretreated with d-amphetamine and air.

This result demonstrates partial blocking of the sensitization process, under the above experimental conditions.

Exposure to nitrous oxide at 75 vol % immediately after pretreatment with d-amphetamine produces significant blocking of the sensitization process, such that the locomotor activity of the animals pretreated with d-amphetamine and with nitrous oxide at 75 vol % induced by the test with d-amphetamine performed at D7 appears significantly lower than that of the animals pretreated with d-amphetamine and with air (P<0.05), but not significantly different from that of the animals pretreated with a saline solution and with nitrous oxide at 75 vol %. Moreover, no “gas” effect was found in the case of the rats pretreated with a saline solution during the acute d-amphetamine test performed at D7, which shows that N₂O blocks the sensitization that is the cause of the addiction and dependency states, but does not affect the acute administration of drug.

Similarly, no significant difference in motor activity was found in response to the saline test at D6, which shows that the gases have no long-term sedative effect.

As regards FIG. 2, it may be seen that, under the above experimental conditions, irrespective of the xenon concentration used, i.e. 50 vol % or 75 vol %, the locomotor activity of the animals pretreated with d-amphetamine and with xenon induced by the d-amphetamine challenge performed on D7 produces blocking of the sensitization to d-amphetamine, such that the locomotor activity of the animals pretreated with d-amphetamine and with xenon appears significantly lower than that of the animals pretreated with d-amphetamine and with air (P<0.05), but not different from that of the animals pretreated with a saline solution and xenon.

As for nitrous oxide (FIG. 1), no significant difference in locomotor activity was found in response to the saline test on D6, which demonstrates in this case also that the gases have no long-term sedative effect.

On the other hand, in the case of the animals pretreated with a saline solution, a significant increase in the response to d-amphetamine is noted in the animals which received xenon at 75 vol %, compared with the animals pretreated with air or xenon at 50 vol %, which might account for a sensitization of the NMDA receptors and a possible toxic effect of xenon at high dose, i.e. around 75% by volume.

Moreover, a histological study of the posterior and retrosplenial cingulate cortices shows, in the case of the rats exposed to xenon at 75 vol %, a generalized cytoplasmic clarification associated with a picnotic appearance of the cell nuclei, and also the appearance in the case of some animals of cytoplasmic vacuoles, which suggest, in accordance with the motor activity, a neurotoxic effect of the repeated administration, for 3 consecutive days, of xenon at 75 vol %.

No similar effect was found in the case of rats exposed to medical air, to nitrous oxide at 75 vol % or to xenon at 50 vol %.

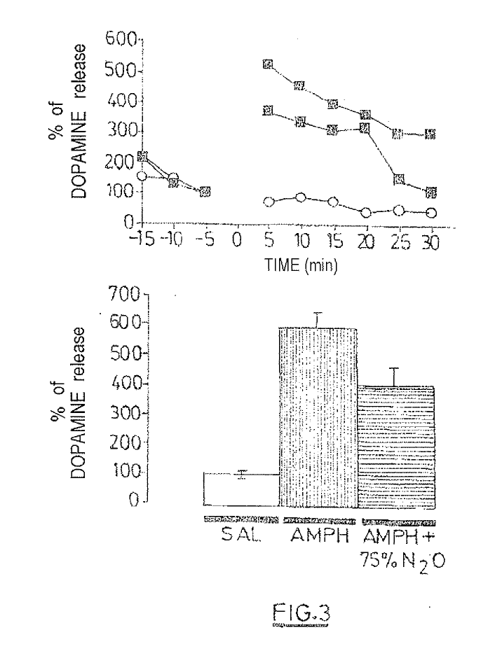

Moreover, FIG. 3 illustrates the effects of nitrous oxide on the increase in dopamine release in the nucleus accumbens septi induced with d-amphetamine. Identical results were obtained with xenon at 50%.

The addition of d-amphetamine at 10⁻⁵ M generates a significant increase in the signal relative to the measured basal signal (P<0.05).

This increase in dopamine release in the nucleus accumbens septi is significantly reduced in the presence of nitrous oxide at 75% or xenon at 50% (by volume) in the infusion liquid (P<0.05).

Without addition of d-amphetamine, the signal remains stable throughout the experiment.

In conclusion, the results obtained clearly show that nitrous oxide and xenon have inhibitory effects on sensitization to d-amphetamine and the dopamine release associated therewith.

Thus, simultaneous exposure of the animals to nitrous oxide or xenon during the phase of sensitization to d-amphetamine totally blocks, in the case of nitrous oxide at 75 vol % or xenon at 50 vol % and 75 vol %, the locomotor hyperactivity due to the sensitization in response to the acute administration of d-amphetamine.

If it is considered that nitrous oxide and xenon show no effect on the glutamatergic receptors of AMPA type (Yakamura and Harris, 2000), their inhibitory effects may be attributed to their antagonist properties on the glutamatergic receptors of NMDA type (Jevtovic-Todorovic et al., 1998; Franks et al., 1998; Yakamura et al., 2000), but also to their antagonist properties on the cholinergic receptors of nicotinic type and to their agonist properties on the GABAergic receptors of A type.

The coadministration of antagonists of the glutamatergic receptors of NMDA type with amphetamines makes it possible to block the sensitization process and the dopamine release associated therewith.

Moreover, the results obtained also show that 75 vol % of nitrous oxide and only 50 vol % of xenon are necessary to block the sensitization process.

However, in the light of both the behavioral and histological effects observed with xenon at high content, a use of xenon at 75% is not recommended.

Specifically, the animals pretreated (from D1 to D3) with a saline solution and xenon at 75% show higher locomotor activity than the control animals pretreated with saline+air, during the d-amphetamine test (performed on D7), which might account for a sensitization of the NMDA receptors.

Moreover, xenon at 75% gives rise to cytoplasmic clarification aggravated, in a few cases, by vacuolization of the neurons of the posterior and retrosplenial cingulate cortices, which unquestionably indicates a neurotoxic process.

In other words, nitrous oxide at 75 vol % or xenon at 50 vol % and 75 vol % block the process of behavioral sensitization to d-amphetamine, but xenon at 75 vol % also induces an increase in the acute response to d-amphetamine, which might reflect a modification in the sensitivity of the receptors involved and a potentially deleterious process, which supports the histological studies.

Furthermore, nitrous oxide and xenon at 50 vol % or 75 vol % block the increase in dopamine release induced with d-amphetamine.

All these results show the incontestable inhibitory effects of nitrous oxide and xenon on sensitization to d-amphetamine and the neurochemical processes associated therewith.

From this, to benefit from the advantages afforded by xenon but without giving rise to the abovementioned deleterious or neurotoxic effects, in particular in the case of more severe neuropathies with an excitotoxic glutamatergic component, and without being penalized by the high cost of this gas, it is thus recommended to use not xenon alone, but rather a gaseous mixture formed from xenon and nitrous oxide, the xenon content needing to be maintained very far from the toxicity threshold of this compound, i.e. typically less than or equal to about 60% in man (i.e. about 75% in rats).

Thus, gaseous mixtures containing from 5% to 35% by volume of xenon gas and from 10% to 50% by volume of nitrous oxide gas (the remainder being oxygen) are entirely suitable for use as gaseous inhalable medicaments for preventing or treating neurointoxications in man or animals.

Specifically, by using suitable mixtures based on xenon and nitrous oxide, it is possible to benefit from the effects of these two compounds without encountering the abovementioned problems.

The gaseous mixture of the invention may be used to treat all neurointoxications. The term “neurointoxication” means a condition, disorder or pathology of the central nervous system whose etiopathogeneity involves, at least partly, an excitotoxic process, especially a dysfunction of excitatory glutamate neurotransmission: see especially the document Parsons et al., Drug News Perspect., 1998, vol. 11, pages 523-569.

Consequently, the treatment not only of the neurotoxic effects of drugs or other substances that can give rise to an addiction, for instance amphetamines and amphetamine derivatives, opiate substances and derivatives thereof, cocaine and its derivatives, tobacco, cannabis and/or alcohol, but also acute cerebral accidents, for instance cranial trauma and cerebralvascular accidents (CVA), including cerebral ischemia; neurodegenerative diseases, for instance Alzheimer's disease, Parkinson's disease, Huntington's disease (chorea), amyotrophic lateral sclerosis, acute disseminated encephalomyelitis, tardive dyskinesia, and olivopontocerebellar degeneration; and various psychiatric or neurological pathologies, such as anxiety disorders, psychotic disorders, especially schizophrenia and epilepsy in its various forms, are included in the context of the present invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A process for preventing or treating a neurointoxication in a human patient, the process comprising administering to the patient via inhalation a medicament in which all or a part of the medicament comprises a gaseous mixture containing xenon gas and nitrous oxide gas, the volume proportion of xenon being between 5% and 45% and the volume proportion of nitrous oxide being between 10% and 50%.

32. The process of claim 31, wherein the neurointoxication results from a cerebral excess of one or more neurotransmitters.

33. The process of claim 31, wherein the gaseous mixture containing xenon and nitrous oxide acts on at least one cerebral receptor so as to reduce the release and/or the effects of dopamine, glutamate, serotonin, taurine, GABA, noradrenalin and/or any other neurotransmitter.

34. The process of claim 31, wherein the remainder of the gaseous mixture is oxygen.

35. The process of 31, wherein the volume proportion of xenon is between 20% and 40% and the volume proportion of nitrous oxide is between 10% and 40%.

36. The process of claim 31, wherein the volume proportion of xenon is between 20% and 32% and the volume proportion of nitrous oxide is between 20% and 40%.

37. The process of claim 36, wherein the volume proportions of xenon and nitrous oxide are each about 30%.

38. The process of claim 31, wherein the volume proportion of xenon is between 10% and 20% and the volume proportion of nitrous oxide is between 40% and 50%.

39. The process of claim 38, wherein the volume proportion of xenon is about 16% and the volume proportion of nitrous oxide is about 50%.

40. The process of claim 31, wherein the medicament also contains oxygen, an oxygen/nitrogen mixture or air, and the gaseous mixture preferably consists of xenon and nitrous oxide, the remainder being oxygen.

41. The process of claim 31, wherein the medicament is ready-to-use.

42. The process of claim 31, wherein the neurointoxication is of the type giving rise to a state of addiction.

43. (canceled)

44. (canceled)

45. (canceled)

46. (canceled)

47. (canceled)

48. (canceled)

49. (canceled)

50. A process for preventing or treating addiction comprising administering to the patient via inhalation a medicament in which all or a part of the medicament comprises a gaseous mixture containing xenon gas and nitrous oxide gas, the volume proportion of xenon being between 5% and 45% and the volume proportion of nitrous oxide being between 10% and 50%.

51. The process of claim 50 wherein the addiction being treated is drug addiction.

52. The process of claim 50 wherein the addiction being treated is addiction to amphetamines and derivatives thereof, cocaine, tobacco, alcohol, cannabis or other dependency-generating substances.

53. A process for the manufacture of all or part of an inhalable medicament for preventing or treating a neurointoxication in a human patient, the process comprising preparing a gaseous mixture that contains xenon gas and nitrous oxide gas with the volume proportion of xenon being between 5% and 45% and the volume proportion of nitrous oxide being between 10% and 50%.
